(19) United States
(12) Patent Application Publication
(10) Pub. No.: US 2013/0096597 A1
(43) Pub. Date: Apr. 18, 2013

(54) REAL-TIME MONITORING AND CONTROL OF HIFU THERAPY IN MULTIPLE DIMENSIONS

(71) Applicants: Ajay Anand, Fishkill, NY (US); John Petruzzello, Carmel, NY (US); Shiwei Zhou, Yorktown Heights, NY (US); Shriram Sethuraman, Briarcliff Manor, NY (US)

(72) Inventors: Ajay Anand, Fishkill, NY (US); John Petruzzello, Carmel, NY (US); Shiwei Zhou, Yorktown Heights, NY (US); Shriram Sethuraman, Briarcliff Manor, NY (US)

(73) Assignee: KONINKLIJKE PHILIPS ELECTRONICS N.V., EINDHOVEN (NL)

(21) Appl. No.: 13/805,396

(22) PCT Filed: Apr. 27, 2011
(86) PCT No.: PCT/IB11/51855

§ 371 Date: Dec. 19, 2012

Related U.S. Application Data
(60) Provisional application No. 61/358,158, filed on Jun. 24, 2010.

(51) Int. Cl. A61N 7/00

(52) U.S. Cl. CPC A61N 7/00
USPC 606/169

(57) ABSTRACT

Energy is transferred (336) to cause a mechanical property of biological tissue to change, as in ablation. An effect of the transferring is examined in more than one spatial dimension to, for example, make an ablation halting decision for a treatment region, i.e., line (312) or layer (314), or for a location (316) within the region. Halting decisions can be based on lesion-central and/or lesion-peripheral longitudinal displacement of treated tissue evaluated in real time against a characteristic curve. Steering in the azimuthal and/or elevation direction is afforded by, for example, linear, or 2D, multi-channel ultrasound arrays for therapy and imaging. Protocols includable are region-wide scanning (SI 010) and location-by-location completion for both (HIFU) therapy and tracking (acoustic-radiation-forced-based) displacement of treated tissue. Fine, location- to-location monitoring can be used for relatively inhomogeneous tissue; whereas, quicker, sparser and more generalized monitoring (1 100, 1200) can be employed for relatively homogeneous tissue.

### FIELD OF THE INVENTION

The present invention relates to transferring energy to cause a mechanical property of biological tissue to change and, more particularly, to examining, in more than one spatial dimension, an effect of the transferring.

BACKGROUND OF THE INVENTION

Tumor ablation therapy using high intensity focused ultrasound (HIFU) has been studied for many years and is just making its way into the United States market and clinical trials.

A tumor, such as a cancer, can be medically treated by surgery and/or chemotherapy. Ablation therapy offers a less intrusive alternative. The ablation may be effected through various alternatives, such as by heating (e.g., radio frequency (RF) ablation, high intensity focused ultrasound (HIFU) ablation, microwave, and laser), freezing (e.g., cryogenic ablation) or chemical action.

HIFU is non-intrusive, in that the thermal energy is applied from outside the body to focus on the tumor, but the energy is not concentrated enough to harm the patient's skin or more internal tissue before it concentrates on the targeted tumor.

Thermal ablation, such as HIFU ablation, raises the temperature at the focal point until the tumor, which may be malignant, is necrosed, i.e., killed, at that ablation point. The necrosed body tissue is known as a lesion. The procedure then moves to another ablation point, and continues point by point until the entire tumor is ablated.

The ablation is guided according to images of the area undergoing treatment. Imaging may be in the form of ultrasound, magnetic resonance imaging (MRI), or x-ray imaging such as fluoroscopy.

MRI is employed for guiding HIFU in ablation, but is expensive. The expense may confine use of this method to research centers worldwide. Also, there exists the potential problem of thermal ablation equipment being MR-compatible.

Acoustic radiation force, by means of ultrasound, has been proposed for monitoring HIFU ablation.

An ultrasound wave imparts to the targeted body tissue a “push” that concentrates at the focal point of the wave. Imaging data before and after the push can reveal information on the nature of the body tissue subjected to the push.

More particularly, tissue necrosed by HIFU therapy, or by other means, at a particular location becomes, at some point, stiffer than untreated tissue. Accordingly, for the same amount of pushing force, less of an axial displacement occurs. The push and subsequent tracking can detect the lessened displacement, and can therefore be used to detect the existence of a lesion formed by ablation.

Lizzi et. al. (“Lizzi”) predicts the use of displacements due to radiation force in real-time HIFU ablation monitoring. F. Lizzi, R. Muratore, C. Deng, J. A. Ketterling, S. K. Alam. S. Mikaelian and A. Kalisz. Ultrasound in Med. & Biol. Vol. 29. No. 11. 1593-1605 (2003).

The Lizzi study proposes that the therapy could be continued until it results in a predetermined alteration in motion characteristics in reaction to pushing.

### SUMMARY OF THE INVENTION

In an aspect of the present invention, it is proposed that conceptualization and implementation of a more fully satisfactory ablation monitoring methodology is needed.

The present invention is directed to addressing the limitations of the prior art in the monitoring of ablation, by providing realization of an accurate, fast, low-cost, simple and convenient technique.

State-of-the-art MRI methods for monitoring HIFU ablation treatment based on temperature are accurate, but require the use of a costly MR suite.

The state of the art in ultrasound guided HIFU (USgHIFU) therapy is to assess the extent of the lesion formed, ablation point by ablation point, after the therapy has been applied.

The time expended in this assessing lengthens the duration of the ablation procedure.

In addition, a typical method is to enter an ablation intensity and a time duration, and then to perform the ablation at the ablation point. However, the instant inventors have observed that treatment time is not a good indicator of lesion size. Thus, the need exists in such a procedure to assess lesion size (and ensure that the desired lesion size has been achieved as per the treatment plan) before moving the therapy focus to the next ablation point.

Furthermore, since the ultrasound solutions that are being used today are not sufficiently accurate in predicting dosage (i.e., duration of HIFU application at the current intensity), the approach is to overdose during treatment to assure necrosis of the entire area.

The Lizzi study predicts the use of acoustic radiation force, an ultrasound technique, in real-time monitoring of HIFU, and the termination of HIFU based on a predetermined alteration of motion characteristics.

However, the Lizzi study does not specify what particular alteration would prudently serve as an indication of when therapy is to be terminated, or when and how the determining of the predetermined alteration is accomplished.

It would be advantageous to have a reliable indicator of when therapy should be halted, one that allows real-time ablation to automatically proceed reliably.

To better address one or more of these concerns, and in accordance with an aspect of the present invention, a previous, commonly-assigned patent application, based on invention disclosure number 776510, entitled “Real-Time Ablation Monitoring for Desired Lesion Size” (hereinafter the “'510 application”) reveals an accurate, fast, low-cost, simple and convenient technique for halting ablation of body tissue at an ablation point.

The instant disclosure continues with and expands upon this methodology. As described in the '510 application, what was proposed is based on evaluating changes along a single axial direction and estimating the lesion's lateral dimensions from that measurement based on an a priori experimentally derived relationship between lateral lesion size and displacement change via the NDD parameter.

In accordance with the present invention, this displacement monitoring is performed in two or three dimensions. For example, multi-element therapy and diagnostic arrays are combinable to control lesion formation in multiple spatial dimensions. Also, displacement monitoring at a particular location may be offset from the therapy focus in an azimuthal and/or elevation direction. Additionally, measures are proposed for reducing the time spent in therapy for cases in which the treatment region is relatively homogeneous so that generalized assumptions can be drawn from a limited amount of such monitoring.

In one version of the present invention, a control device is provided for a unit that issues a beam for changing a mechanical property, such as stiffness, of biological tissue. The device applies an acoustic-radiation-force-based push beam whose focus is, in an azimuthal and/or elevation direction, offset from the most recent focus of the mechanical-property-changing beam.

In an aspect, the offset is to a target periphery of a lesion created by the mechanical-property-changing beam with that most recent focus.

The mechanical-property-changing beam, in a further aspect, is maintained at a current location until determining that treatment at the current location is completed.

In one embodiment, the mechanical-property-changing beam is repeatedly interspersed with a push beam and the tracking beam in real time. Based on the determining, regarding location-dependent treatment completion, in real time, scanning occurs in real time from the current location within a treatment region within the tissue to the next location within the region.

In an additional version, a control device for a unit for transferring energy for causing a mechanical property of biological tissue to undergo change includes a multi-channel ultrasound transducer array. The array is configured for electronically steering a tracking beam in an azimuthal and/or elevation direction. The tracking is of displacement caused by a push to the tissue to assess an effect of the energy transfer.

In a variation upon this aspect, the array is two-dimensional and configured for the steering in both the azimuthal and elevation directions.

In one further aspect, the displacement is applied to a characteristic curve to predict lesion size.

In another aspect, during an interruption in the energy transfer, the tracking beam is steered from location to location within a treatment region within the tissue.

According to a particular aspect, prior to introducing thermal effects into a treatment line or layer within the tissue by means of the energy transfer, a baseline is created usable in decisions on whether treatment at locations within the line or layer are completed, the creating being based on results from scanning the line or layer with pushes and tracking pulses.

In yet another aspect, it is determined that a location within a treatment region within said tissue is no longer to be treated with a beam by means of which the energy transfer occurs.

In accordance with a related, different aspect, the steering, the tracking and the determining are performed in real time.

In a further related aspect, the steering, the tracking, the determining, and deciding that treatment of the region is completed are performed automatically and without need for user intervention.

As one other aspect, the control device is configured for steering a push beam from location to location within a treatment region within the tissue during an interruption in the energy transfer.

In a different but related aspect, the tracking beam is offset from the push to a target periphery of the lesion currently being formed.

In a supplementary aspect, the unit being controlled includes a multi-channel ultrasound transducer array configured for steering in an azimuthal and/or elevation direction a beam by means of which the energy transfer occurs.

In an additional version, a device is configured for scanning a beam for changing a mechanical property of biological tissue within a treatment region and for monitoring displacement at a particular location within the region as representative of the region.

In a related sub-aspect, while the monitoring is not performed, scanning location by location is performed in runs that are repeated, skipping locations for which it has been determined that treatment is completed.

In an alternative sub-aspect, when it is determined that mechanical-property-changing treatment to a current location within the region is no longer to be applied, the scanning is performed to a next location if a next location is to be treated, and, without need for any pushing or any tracking, treatment is repeated at the next location which now serves as the current location for purposes of any further repetition.

In a particular version, a control device for a unit configured for issuing a beam for causing a mechanical property of biological tissue to undergo change performs mechanical-property-changing beam scanning to repeatedly span a treatment region within the tissue. The scanning skips any location that has been determined to no longer to receive treatment. Scanning also occurs by means of a beam for tracking, during an interruption of the treatment, at least one unfocused push to the region.

Details of the novel ablation control are set forth further below, with the aid of the following drawings.

### BRIEF DESCRIPTION OF THE DRAWINGS

FIG. 1 is an exemplary functional diagram of an ablation system;

FIG. 2 is one type of suggested signal timing scheme;

FIG. 3 is an example of how a baseline of initial displacement values is obtained for use in assessing the progress of ablation throughout a treatment region;

FIG. 4 is one example of a graph of a typical displacement over time in units of monitoring/therapy cycles, and of a quadratic curve fitted to an initial portion of the graph for peak detection;

FIG. 5 is an exemplary graph of normalized displacement over time;

FIG. 6 is an example of a graph of lesion diameter versus normalized displacement difference;

FIG. 7 is a flowchart of an example of preparation and initialization of an ablation control device;

FIG. 8 is an illustration depicting an example of the focus of a push being offset from the focus of the therapy beam whose effect is being measured;

FIG. 9 is a flow chart demonstrating an exemplary real-time procedure for, automatically and without the need for user intervention, finely monitoring ablation that is performed one location at a time;

FIG. 10 is a flow chart demonstrating an exemplary real-time procedure for, automatically and without the need for user intervention, finely monitoring ablation that is performed one location at a time;

FIG. 11 is a flow chart of a real-time procedure for, automatically and without the need for user intervention, time-efficient monitoring of a relatively homogeneous treatment region from a single location representative of the entire region; and

FIG. 12 is a flow chart exemplifying a real-time procedure for, automatically and without the need for user intervention, time-efficient monitoring of a treatment region exhibiting a certain degree of homogeneity.

### DETAILED DESCRIPTION OF EMBODIMENTS

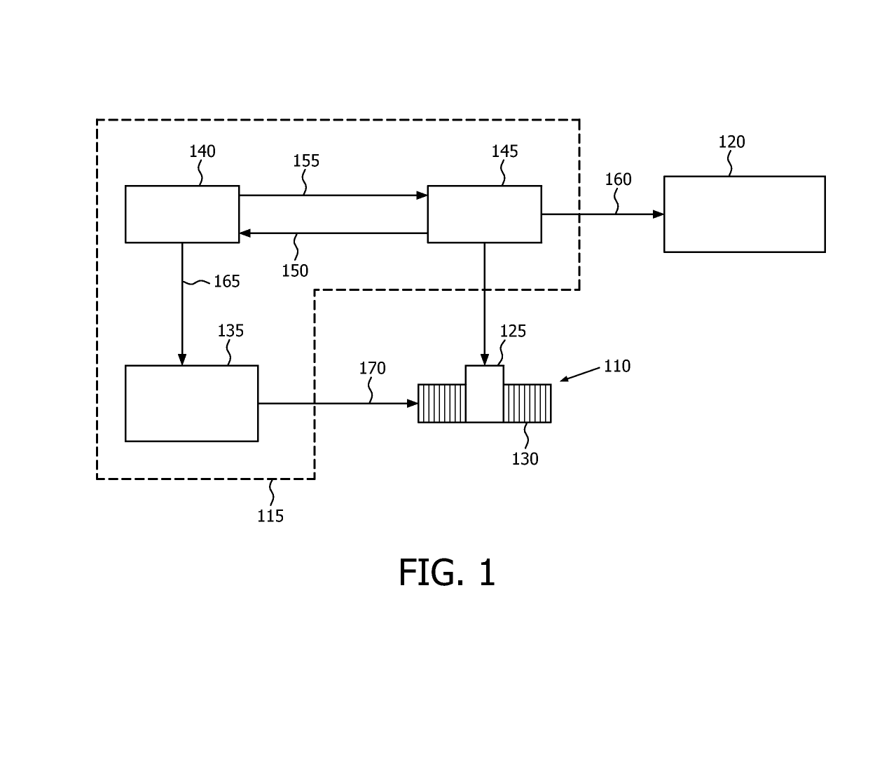

FIG. 1 depicts, by way of illustrative and non-limitative example, a mechanical-property-changing, or “ablation,” unit 110, its control device 115 for monitoring therapy in multiple spatial dimensions, and a real-time display 120.

The ablation unit 110 includes a multi-element diagnostic array 125 placed confocally with a therapeutic or “therapy,” array 130.

The control device 115 comprises a combination multi-channel high power amplifier and matching network module 135, a triggering and control logic module 140, and a multi-channel ultrasound data acquisition and analysis module 145. The control device 115 may be implemented as, for example, an electrical unit, analog electronic components, a hybrid circuit, or a solid state device comprising an integrated circuit which includes any form of RAM, ROM, ASIC, PLD, or combination thereof. The modules 135, 140, 145 may each be implemented in software, firmware or hardware or a combination thereof.

The therapy array 130 is implementable as a high intensity focused ultrasound (HIFU) transducer, and, like the diagnostic array 125, may be implemented as, for example, a linear array, phased array or two-dimensional (2D) matrix transducer. The HIFU transducer 130 focuses ultrasound (which is radio frequency or “RF” energy) to thereby ablate the tumor or other target of ablation. The HIFU transducer 130 also delivers ultrasound in the form of an acoustic radiation force imaging (ARFI) push, and receives back the echoes from the ablation subject. The term “ablation subject”hereinafter refers to the medical patient receiving therapy, whether human or animal, or any body tissue such as when testing is conducted. The arrays 125, 130 are housed in a probe (not shown) to be placed on the patient by computer control or manually. Alternatively the probe may be placed at the end of flexible shaft to be introduced internally, as by the mouth of a patient under anesthesia. The probe may contain the beamforming circuitry or the circuitry may reside in the triggering and control logic module 140.

The driving signals for the therapy array 130 are provided by the multi-channel high power amplifier/matching network module 135.

Control logic of the control device 115 is employed to provide triggering and control signals to synchronize the timing of three types of acoustic beams which are interspersed. Firstly there are mechanical-property-changing, or “therapy,” beams, from the therapy array 130, for changing a mechanical property of biological tissue. Secondly, there are push beams, from the therapy array, for assessing the effect of the therapy beams. Thirdly, there are tracking beams, from the diagnostic array 125, for, in making the assessment, tracking tissue displacement due to the push. The triggering may be gated to follow a particular snapshot in time of the heartbeat and/or respiration cycles depending on the location of the in vivo ablation site being subject to ablation. Associated with the control logic is a graphical user interface (GUI) having user interface input/output means that may include keys, dials, sliders, trackballs, touch-sensitive screens, cursors and any other known and suitable actuators for specification of treatment boundaries and parameters. The control logic is realizable in the form of a PC-based software program, e.g., LabVIEW™ based.

The multi-channel ultrasound data acquisition and analysis module 145 interfaces with the diagnostic array 125 to process the backscattered signals to thereby compute the change in mechanical displacements. The computation serves as a measure of stiffness to thereby detect completion of therapy at the current location being treated. Lesion dimensions based on the ongoing computation can optionally be displayed on the real-time display 120 as an image and/or superimposed on a B-mode image.

A control signal 150 is also fed from the multi-channel ultrasound data acquisition and analysis module 145 to the triggering and control logic module 140 to, based on the monitoring analysis, stop therapy when the desired treatment endpoint for the current location or the treatment region has been reached.

The other arrows 155, 160, 165, 170 indicate the controlling relationship in accordance with the above discussion.

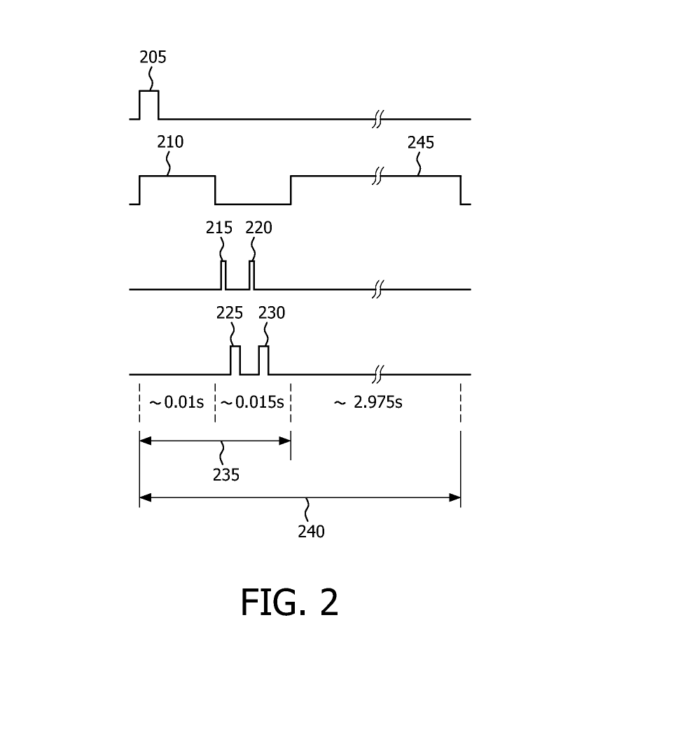

FIG. 2 illustrates one scheme for the synchronization of push, tracking, and therapy pulses of the respective beams in the ablation control device 115. In the exemplary embodiment shown, a master trigger 205 is followed by a push 210 from the HIFU transducer 130. The push duration is set for between 10 and 15 milliseconds (ms), depending on mechanical properties of the tissue to undergo ablation. Following the push 210 are first and second tracking pulses 215, 220 emanating from the diagnostic array 125. The tracking pulses 215, 220 are employed to perceive structures at different depths along the receive line in the body tissue. The first tracking pulse 215 issues immediately after the push 210 to interrogate the strained tissue value. The second tracking pulse 220 issues about 12 ms later and represents the relaxed (or equilibrium) tissue value. The multi-channel ultrasound data acquisition and analysis module 145 records corresponding return echoes 225, 230 of these two tracking pulses 215, 220 immediately following each of the two pulses. Differences between the RF data retrieved from these two return echoes 225, 230 represent the displacement the body tissue has undergone in reaction to the push 210. This entire sequence is a monitoring portion 235 of a monitoring-therapy cycle 240, and lasts between 20 and 30 ms. The therapy portion 245, during which the HIFU transducer 130 delivers therapy, is much larger, and lasts between 2970 and 2980 ms. Consequently, the entire monitoring-therapy cycle 240 lasts for about 3 seconds.

Other possible timing sequences can be substituted for the one in FIG. 2, such as where the first tracking pulse 215 precedes the push and the second tracking pulse 220 occurs after the push. As in FIG. 2, the spatial position revealed as a result of the first tracking pulse 215 is compared to the spatial position revealed as a result of the second tracking pulse 220 to derive the displacement resulting from the push. As a further example, monitoring may be simultaneous with pushing. Also, the displacement induced may be oscillatory, as with harmonic motion imaging (HMI).

Due to the focused nature of the ultrasound beam being applied in the push 210, displacement is maximal at the focus. However, displacement to lesser extents occurs axially and radially away from the focus. The displacement is affected, over time, by the heat delivered by the therapy ultrasound beam from the HIFU transducer 130.

To take advantage of a larger and more noticeable displacement, and for uniformity in measuring ablation point-to-ablation point, it is desirable to focus the beam delivering the push 210 at the focus of the therapy ultrasound beam (or “therapy focus”) so that the two foci coincide. The two beams emanate from the same HIFU transducer 130. Although the therapy beam is at a higher power than the push beam, the two beams share the same focusing parameters and the same focus (or “focal point”).

The tracking pulses 215, 220 originate from a separate array 125 than that producing the push/therapy focus; however, the two arrays 125, 130 can be configured in fixed spatial relation, one placed confocally with the other.

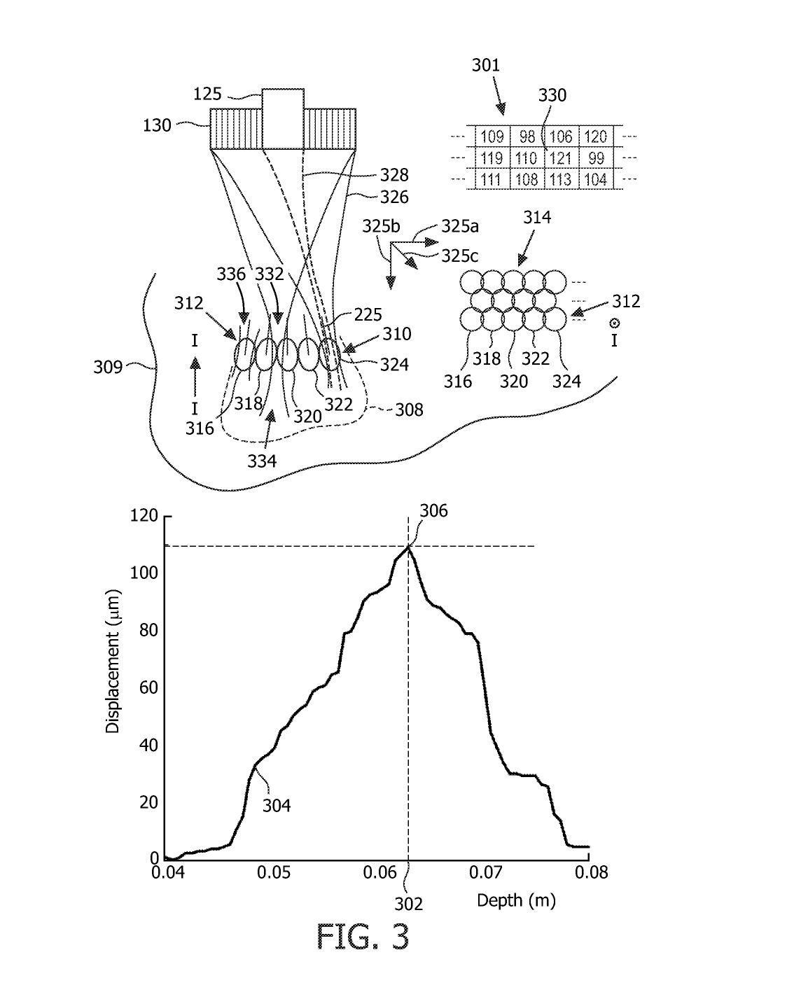

FIG. 3 is an example of how a baseline 301 of initial displacement values 306 is obtained for use in assessing the progress of ablation throughout a treatment region. The FIG. 3 graph represents displacements 304 along a receive line 225. What is termed an “initial displacement” 306 is the maximum of the displacements 304 along the receive line 225, all resulting from a push 210 at a single location of a pre-therapy baseline scan. Moreover, because the receive line 225 is aligned with the push beam, the location of the initial displacement 306 is not only the location of the spatially maximum displacement along the receive line, but an estimate of the spatially maximum displacement in three-dimensional space. Since the push and therapy beams are confocal, the therapy focus 302 coincides with the location of the initial displacement 306.

Before treatment begins, B-mode imaging can be used to display a treatment volume 308 on-screen, so that the clinician can define the target tissue, e.g., by drawing an on-screen boundary. The treatment volume 308, within biological tissue 309, includes one or more treatment regions 310. The treatment region 310 includes one or more treatment lines 312 each a single row, or treatment layers 314 each having multiple side-by-side rows, of lesions 316, 318, 320, 322, 324 . . . To the side of FIG. 3 is shown a top view (as indicated by arrow “I” here normal to the drawing sheet) of the treatment region 310 in the 3D steering case. A portion of the top layer 314 is shown. If the arrays 125, 130 are configured for 2D steering, the line 312 is scannable in the azimuthal 325a or elevation 325b direction, and the arrays can be mechanically translated to treat any laterally adjacent line. If, on the other hand, the arrays are configured for 3D steering, the layer 314, and any underlying layer, is scannable in the azimuthal 325 a and/or elevation 325b direction.

The therapy array 130, if it is a linear array for example, is configured for electronically steering the therapy beam 336 and the push beam 326 in the azimuthal direction 325a. If the therapy array 130 is, instead, a 2D array, it is configured for electronically steering the therapy beam 336 and push beam 326 in the azimuthal direction 325a, the elevation direction 325b or in a combination 325c of the two directions.

Likewise, for the diagnostic array 125, if it is a linear array, it is configured for electronically steering the tracking beam 328 of pulses 215, 220 in the azimuthal direction 325a. If the diagnostic array 125 is, instead, like the therapy array 130, a 2D array, it is configured for electronically steering the tracking beam 328 in the azimuthal direction 325a, the elevation direction 325b or in a combination 325c of the two directions.

A baseline is an array of acquired initial displacements 306, the array being correspondingly one-dimensional in the case of the line 312, and two-dimensional in the case of the layer 314. For embodiments in which the lesions 316-324 are formed one by one, real-time treatment of one line 312 or layer 314 can proceed to baseline acquisition for a next, e.g., underlying or overlying, line or layer with little or no pausing for thermal effects to dissipate. For embodiments in which the lesions are formed concurrently, the next line 312 or layer 314 can be a non-adjacent one to shorten or avoid the pausing.

The clinician may also enter a lesion size, which can be in the form of a normalized displacement difference which is discussed further below. Alternatively, the lesion size is set automatically.

For baseline acquisition, a push beam 326 at a starting location 324 is followed by a tracking beam 328 of pulses 215, 220. The longitudinally coincident respective receive lines 225, 230 (only line 225 being shown in FIG. 3) for the pulses 215, 220 are cross-correlated to measure displacement, the maximum of which is the initial displacement 306. The push beam 326 and the tracking beam 328 are then scanned to the next location 322, and the procedure is repeated.

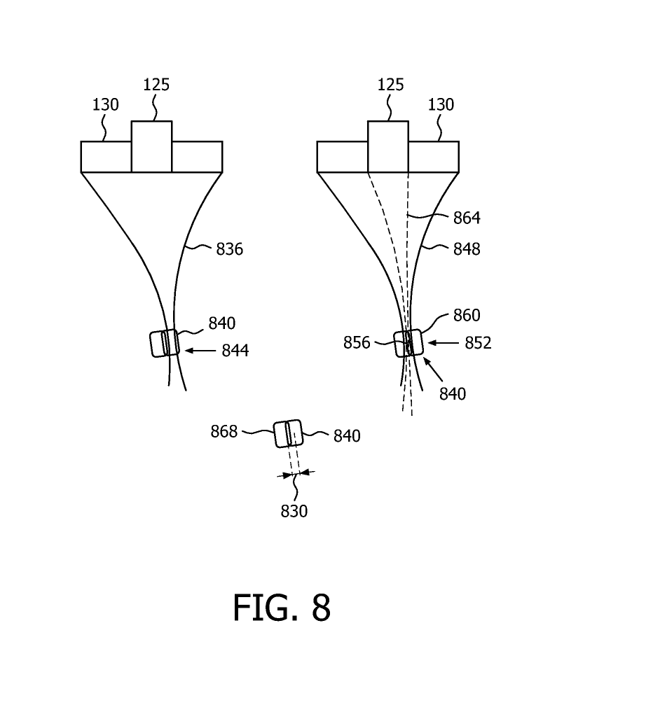

In some embodiments, a baseline value 330 is obtained for an intermediate location 332, at a target periphery of the lesion 320 where it is predicted to meet an adjacent lesion 318. A push beam 334 subject to tracking focuses at the meeting location 332. This is done for refinement or “fine-tuning” of lesion size, as discussed further below in connection with FIG. 8.

The baseline value, and/or intermediate baseline value at the target periphery, for example, of a location 320 is usable in deciding when treatment with the mechanical-property-changing, or “therapy,” beam 336 at that location is completed, as discussed immediately below.

FIG. 4 is an example of a graph of a typical displacement over time in units of monitoring/therapy cycles 240, and of a quadratic curve fitted to an initial portion of the graph for peak detection. Cycle number zero, in the graph, refers to the start of the monitoring-therapy cycles 240. In the example of FIG. 4, a starting displacement 405 is shown to be about 110 μm. The starting displacement 405 varies from ablation point to ablation point, individual to individual, and tissue sample to tissue sample, because of the inhomogeneities of the body tissue. Going forward in time, with each successive monitoring-therapy cycle 240, measurement is made of the effect 407, or “thermal effect,” of the therapy beam 336, at the current location 316 within the treatment region 310 within the tissue 309, on the tissue displacement 410 at the therapy focus 302. The displacement 410, by means of the pushes during the push portion 210, initially increases over time, due to the applied heat softening the tissue. After some therapy time, the displacement 410 reaches a peak 415 and starts to decrease, indicating that the tissue is becoming stiffer (i.e., upon necrosis). The decrease is observed until the therapy reaches a stopping point in the displacements 410 or “endpoint displacement” 420. After the therapy is turned off, the displacement 410 decrease slows down as the tissue is cooling. However, the effect of temperature on cell necrosis still exists, even though a transfer of energy is no longer being applied, e.g., by means of a beam, to change a mechanical property of biological tissue.

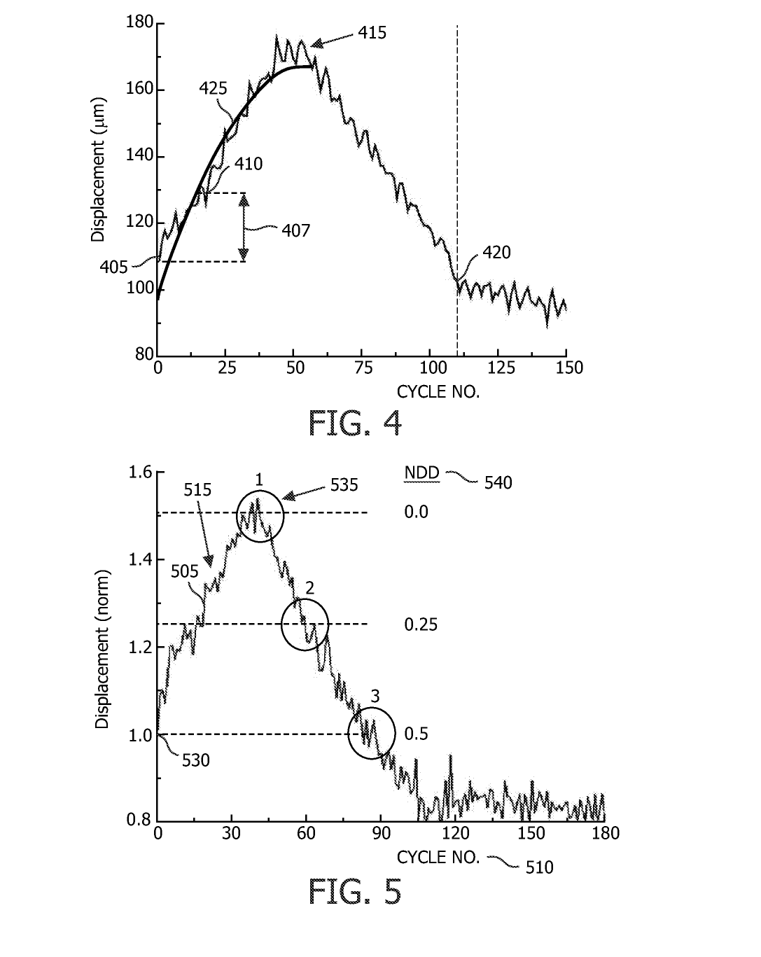

A quadratic curve 425 may be fitted to the displacements 410 in real time to detect the peak 415. The peak 415 is detected when the slope of the quadratic curve 425 becomes zero and starts to turn negative. The peak 415 may be estimated by averaging displacement 410 measurements, e.g., for five cycles, within an interval around the zero slope point. A reason for detecting the peak 415 will be discussed in detail below in connection with FIG. 5.

FIG. 5 is an exemplary graph of normalized displacement 505 over time, or, more specifically, according to cycle number 510. The FIG. 5 graph, termed hereinafter a characteristic curve 515, can be derived from the displacement graph of FIG. 4 by dividing each displacement 410 by the starting displacement 405. The word “characteristic” in the term “characteristic curve” as used herein refers to a distinguishing feature or attribute. The distinguishing feature or attribute may pertain to body or biological tissue. The characteristic curve 515 may also be a combination, such as an average, of a number of such derived curves, based on empirical observation at different ablation points. Due to the above-noted inhomogeneities of body tissue, the FIG. 5 time scale (of cycle numbers 510) may shrink or expand, depending on the ablation point, individual or tissue sample. Thus, the time rate of normalized displacement is variable. However, the shape of the characteristic curve 515 remains constant for a given type of body tissue, e.g., liver, breast, heart. By implication, once a point on the characteristic curve 515 has been identified, all points are identified. This is significant, because some of the points on the characteristic curve 515 are associated with specific lesion sizes. Thus, the ability to identify that an ongoing ablation at an ablation point has reached a specific point on the characteristic curve 515 can lead to an accurate prediction 540, i.e., at an NDD of 0.5 for example, of when to halt the ablation to achieve a desired lesion size. The prediction 540 is here based on a “central” NDD, the NDD at the therapy focus 302. However, the NDD parameter derived from a push beam focus for assessing the effect of the most recent therapy beam focus 302 can be offset, in an azimuthal 325a and/or elevation 325b direction. The offset would be to, for example, a predicted meeting point 332 on the target periphery of a lesion 320. The “peripheral” NDD can be used, or contribute, to a real-time decision that treatment at the current location 320 is completed. A “peripheral” NDD of 0.1 to 0.15, for example, which could imply sufficient progress in the onset of necrosis at the predicted meeting point 320 with what will be the next, adjacent lesion 318, may indicate that treatment is completed at the current location 320.

During the current ablation, the pre-normalized displacements 410 are available in real time. A technique discussed in the commonly-assigned '510 application is to register one or more displacements 410 with the associated normalized displacement(s) 505 of the characteristic curve 515.

Two landmark points on the characteristic curve 515 are the normalized starting displacement 530, which by convention is set to unity, and the normalized peak displacement 535.

The associated pre-normalized displacements are, respectively, the starting displacement 405 and the peak displacement 415.

More specifically, the starting displacement 405 may be registered to the starting normalized displacement 530. The registration allows, by means of the characteristic curve 515, the starting displacement 405 to be utilized in predicting when, displacement-wise, ablation should be halted to achieve a predetermined lesion size upon halting. The starting displacement 405 is accordingly one of the values that can serve as what is termed hereinafter a therapy-progress-rate-independent (TPRI) registration point, as discussed in detail further below.

The peak displacement 415 occurs simultaneously with the normalized peak displacement 535. Accordingly, the peak displacement 415 can, like the starting displacement 405, serve as a TPRI registration point.

For its effectiveness as a predictor of lesion size, registration of the TPRI registration point to the characteristic curve 515 relies on a functional relationship between decrements in normalized displacement 505 and empirical values of lesion size. For this purpose, a normalized displacement difference (NDD) 540 is defined as the difference between the normalized peak displacement 535 and an endpoint of the normalized displacement 505. NDD 540 values of 0, 0.25 and 0.5 are shown in FIG. 5. Thus, for example, with an NDD equal to 0, the normalized peak displacement 535 and the normalized endpoint displacement 505 are the same, which would imply that the application of ablation energy is halted at peak displacement 415 (or, equivalently, at normalized peak displacement 535). A particular lesion size is associated with each value of the NDD 540.

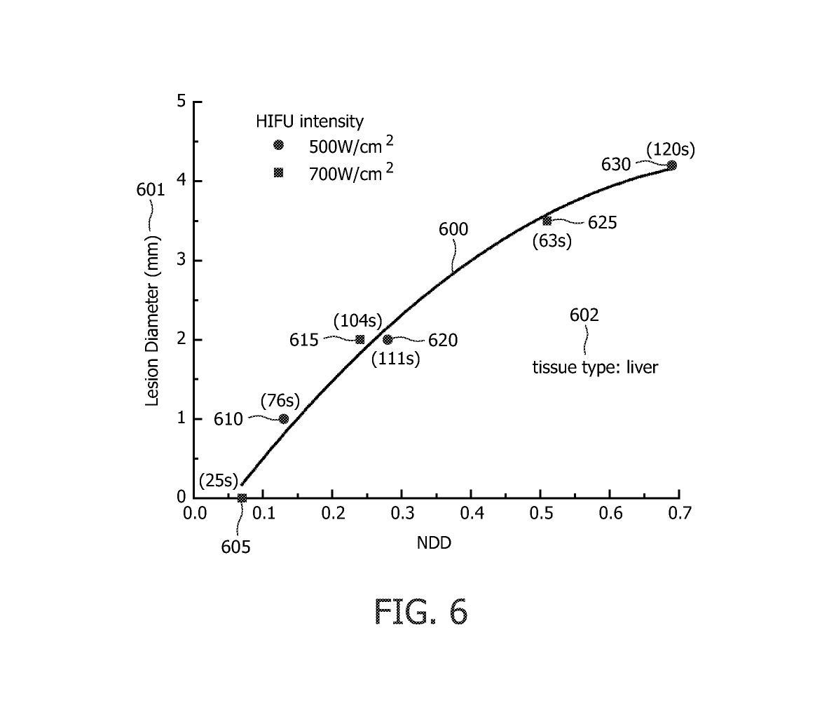

FIG. 6 is an example of a graph 600 of lesion diameter versus NDD 540. Ablation was conducted experimentally on various tissue samples and various sites within a sample. The ablation was halted, and the sample was immediately cooled to stop necrosing. The size of the lesion was measured. The lesion shape depends on the transducer geometry and its acoustic beam characteristics. In the case of HIFU, the lesion shape is commonly ellipsoidal with the major axis along the beam's longitudinal center. The lesion diameter in FIG. 6 accordingly refers to the maximum lesion diameter perpendicular to the beam's longitudinal center. For each measurement, the treatment time, endpoint displacement value 420 and peak displacement value 415 were noted. Based on this actual data, observation points were plotted, relating lesion diameter to NDD 540. FIG. 6 shows some plotted observation points for the tissue type 602, which in this case is liver. It was found that the relationship is described by a second order polynomial fit with good agreement, and that the parameters of the polynomial vary with tissue type. The parameters would also vary with lesion shape, although lesion shape would not typically be varied. It is therefore assumed hereinafter that, when curves are classified by tissue type, there exists no need to further classify by lesion shape. As shown by the different HIFU intensities of the observations 605-630, the fitted function is invariant with treatment intensity. The treatment times for the six samples are listed in parentheses. It can be seen that the treatment time is not a good indicator of lesion size, due to inhomogeneities of the tissue. Observation 615, for example, indicates more treatment time to achieve a smaller lesion size in comparison to observation 625. For observations made for different parts of the same tissue sample or for different tissue samples, lesion sizes have been found not to correlate well with treatment time. Advantageously, methodology of the '510 application, as also set forth hereinabove and in more detail below, overcomes sensitivity to tissue inhomogeneity.

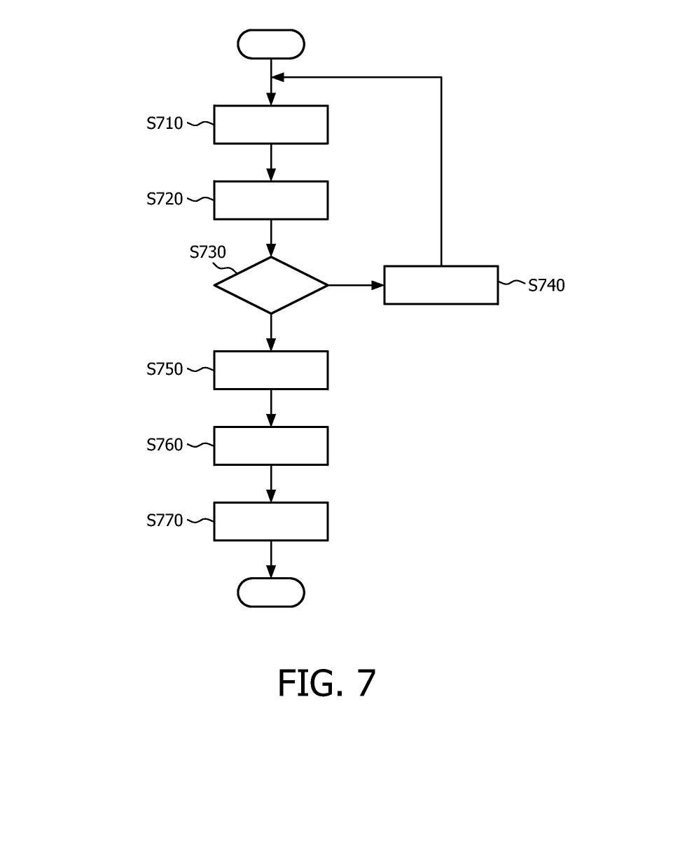

FIG. 7 provides an example of preparation and initialization of the ablation control device 115. Ablation is performed on a particular tissue sample (step S710). Ablation is terminated for the current tissue sample, which is immediately cooled to stop necrosis. Endpoint displacement 420 and peak displacement 415 have been recorded. After histological examination of the lesion formed, the lesion size is recorded (step S720). Query is then made on whether this is the last observation (step S730). If it is not the last observation, a next observation is made, on the current tissue sample or another tissue sample or on another tissue type (step S740). On the other hand, if it is the last observation, the observations are grouped by tissue type (step S750). Fitted curves 600 (or “calibration curves”) are derived by tissue type, using the recorded data and quadratic curve fitting (step S760). The calibration curves 600, each with its identifier of tissue type 602, are sent to the ablation control device 115. Also, each characteristic curve 515, identified by tissue type, is made available to the ablation control device 115. The characteristic curves 515 have, likewise, been derived from empirical observation, as mentioned above (step S770).

Once the baseline 301 is acquired, the therapy beam 336 is applied, and interrupted to execute one or more monitoring portions 235 for respective locations 316-324, depending on the protocol, as discussed in detail further below. The interruptions to therapy occur interleavingly to allow each time for the one or more monitoring portions 235. In the monitoring of push-induced displacements 410 at a given location, one or more TPRI registration point(s) are obtained, in real time, and processed, in real time. The processing involves registering the point(s) (e.g., starting displacement 405, peak displacement 415) to the corresponding point(s) (i.e., normalized starting displacement 530, normalized peak displacement 535) on the appropriate characteristic curve 515. The following formula may be used:

HD=(NPD−NDD)×RP/CP [formula (1)]

where

HD stands for the displacement upon which ablation is to be halted;

RP stands for TPRI registration point;

CP stands for the corresponding point of the characteristic (i.e., normalized) curve 515;

NPD stands for normalized peak displacement 535; and

NDD stands for normalized displacement difference 540.

Thus, the determining of the HD, i.e., endpoint displacement 420, is enabled by the registering of the TPRI registration point(s) with the characteristic curve 515. Therefore, for example, if the starting displacement 405 serves as the TPRI registration point, the enabling occurs upon completion of the monitoring portion 235 of the first of the monitoring-therapy cycles 240. Prior to that completion, the starting displacement 405 is not yet known, and therefore cannot be applied as RP in formula (1) shown above.

The quantity RP/CP in formula (1) may be regarded as a normalization factor. When the desired lesion size is evaluated against the calibration curve 600, the NDD 540 is identified. The NDD 540 is subtracted from the NPD 535 to yield the normalized form of the endpoint displacement 420. This normalized form is multiplied by the normalization factor to yield the “de-normalized” endpoint displacement (or HD in formula (1)). If more than one registration point is used, the corresponding normalization factors can be averaged for use in equation (1).

FIG. 8 depicts, as an illustration, the focus of a push being offset 830 from the focus of the therapy beam whose effect is being measured.

A therapy beam 836 is applied to a location 840 and is kept positionally fixed at the location. During an interruption in the therapy beam 836 having a focus 844, a push beam 848 having a focus 852 is applied to a point 856 on a target periphery 860 of the lesion 840 created by the therapy beam 836 having a most recent focus 844. The focus 852 of the push beam 848 is for assessing the effect of the most recent focus 844 of the therapy beam 836, the foci 844, 852 being offset 830 in at least one of an azimuthal direction and an elevation direction. The push beam 848 is followed by a pair 864 of first and second tracking pulses to image the tissue 309 in its strained and relaxed position, respectively. As mentioned above in relation to FIG. 3, a “peripheral” NDD of 0.1 to 0.15, for example, which could imply sufficient progress in the onset of necrosis at the predicted meeting point 856 with what will be the next, adjacent lesion 868, may indicate that treatment is completed at the current location 840.

Alternatively, instead of offsetting, from the therapy beam 836 both the push and the tracking, the tracking alone, for example, can be offset. Baseline acquisition, accordingly would include initial displacements based on “lesion-central” pushes but tracking pulses 210, 215 aligned according to the offset 830. Consequently, in FIG. 8, the push 848 would be aligned not with the predicted meeting point 856, but centrally with the current location 840. The tracking beam 864, however, would remain aligned according to the offset 830. Likewise during therapy, the push beam is aligned centrally with the lesion 840; whereas, the tracking pulses 864 exemplified in FIG. 8 are aligned according to the offset 830, as shown.

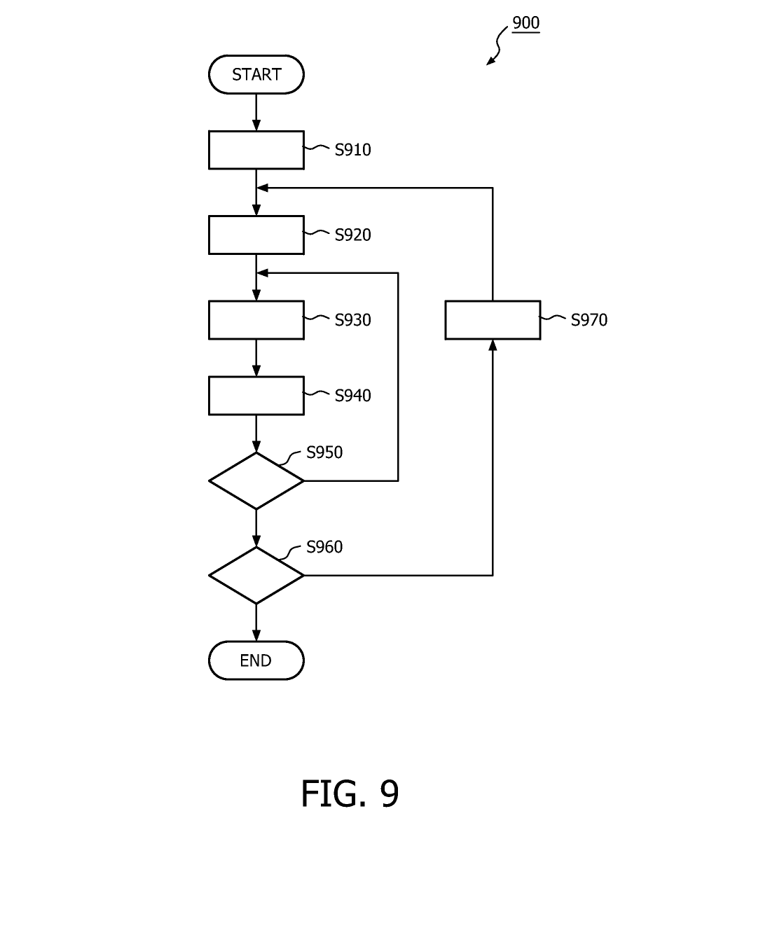

FIG. 9 demonstrates a real-time procedure 900 for, automatically and without the need for user intervention, finely monitoring ablation that is performed one location 840 at a time. Initially, the baseline 301, usable in decisions on whether treatment at a location 840 is completed, is acquired (step S910). The therapy beam focus 844 is maintained at the current location 840 (step S920). The therapy beam 836 issues (step S930). The therapy beam 836 is interrupted, i.e., the therapy portion 245 of the monitoring-therapy cycle 240 is concluded, after, for example, about 3 seconds or a specific number of cycles, to issue the push beam 848 and the pair 864 of tracking pulses (step S940). If it is determined that treatment at the current location 840 is not yet completed (step S950), processing returns step S930. Otherwise, if it is determined that treatment at the current location 840 is completed and that the current location is therefore no longer to be treated, and there is a next location in the treatment region 310 (step S960), beamforming logic for the therapy array 130 steers to scan to the next location 868 (step S970), which becomes the current location for purposes of further repetition. Processing returns to step S920. If, on the other hand, treatment in the treatment region 310 is complete (step S960), the procedure ends.

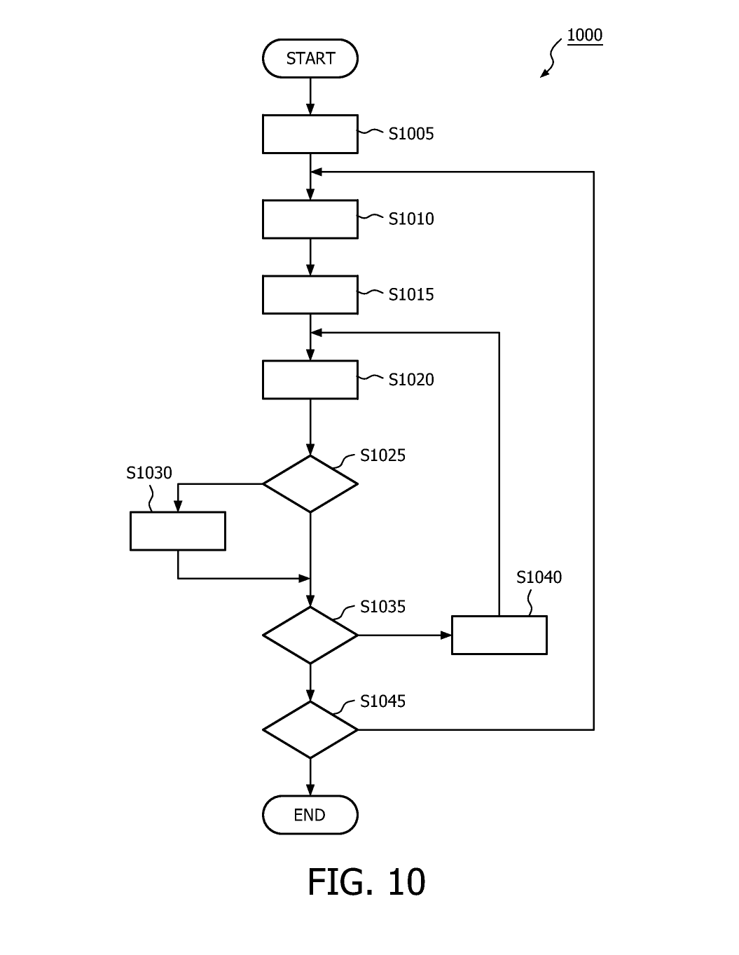

FIG. 10 shows a real-time procedure for, automatically and without the need for user intervention, finely monitoring ablation that is performed concurrently throughout the treatment region 310. First, the baseline 301 is acquired (step S1005). The therapy beam 836 then continually scans the treatment region 310 location by location in runs that are repeated, but skipping recorded locations 316-324 . . . for which treatment is completed. Each run, but for the skipping, spans the region 310. In the layer 314, for example, the bottom-most locations 316-324 (from the top view perspective) can be part of a left-to-right sweep, such a sweep then proceeding upward row by row to constitute a single run. The scanning continues until the treatment is interrupted, e.g., by expiry of an approximately 3 second time period (step S1010). The first location 316 becomes the current location (step S1015). The push beam 848 and the pair 864 of tracking pulses 215, 220 issue (step S1020). If it is decided that treatment for the current location is completed (step S1025), the location is recorded (step S1030). If, skipping the recorded locations, there is a next location within the treatment region 310 (step S1035), beamforming logic for the therapy array 130 steers to scan, i.e., the push beam 848 and the tracking beam 328 of pulses 215, 220, to that next location (step S1040) and processing returns to step S1020. Otherwise, if monitoring has reached completion for the current interruption in therapy, and if treatment of the treatment region 310 is not yet completed (step S1045), processing returns to step S1010.

The above-described monitoring schemes 900, 1000 are useful clinically where presence of tissue heterogeneities and/or blood vessels can result in locations 840 within a treatment line 312 or layer 314 reaching necrosis faster than others for the same applied therapeutic power. In such cases, the monitoring techniques in the above-described procedures 900, 1000 would help optimize the therapy delivery, reduce over treatment and thereby also treatment duration. In addition, based on the thermal diffusion process, for the same amount of heat applied across the scanned line 312 or layer 314, the temperature rise at the extremities would ordinarily be lower than that at the center, owing to the strong thermal gradient at the edges. Hence, the center would need to be treated less than the extremities. The monitoring protocols of the above-described monitoring procedures 900, 1000 are designed to provide feedback to continue or stop the therapy accordingly.

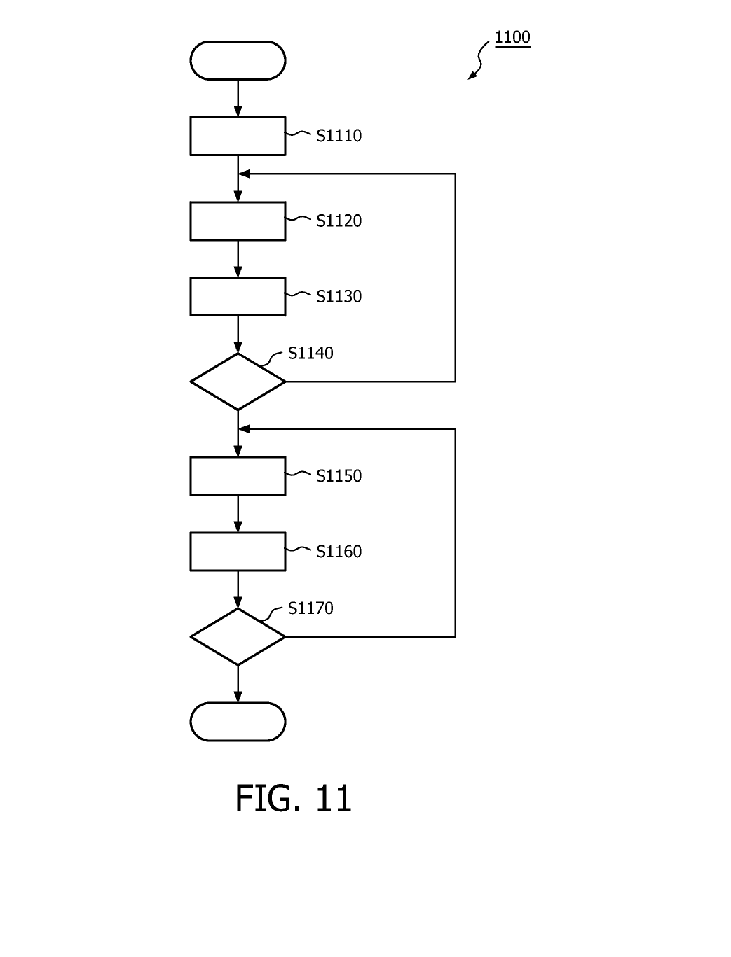

FIG. 11 shows a real-time procedure 1100 for, automatically and without the need for user intervention, time-efficient monitoring of a relatively homogeneous treatment region 310 from a single location 316 representative of the entire region. A baseline value 330, or “initial displacement value” 306, for a particular location 332 to be tracked is acquired (step S1110). Therapy is applied either to the particular location 316, or to the treatment region 310 by continually scanning it repeatedly run by run. In either event, the therapy continues until interrupted, as by time expiry (step S1120). The push beam 848 and the pair 864 of tracking pulses 215, 220 issue to the single, particular location 316 (step S1130). If the treatment, as judged by monitoring of the single, particular location 316, is not yet completed (step S1140), processing returns to step S1120. Otherwise, if treatment as so judged, is determined to have been completed (step S1140), beamforming logic for the therapy array 130 steers to scan to the next location which becomes the current location for purposes of repetition (step S1150). Treatment is applied to the current location for the same duration as it was applied to the particular location 316, without the need now for any pushing or tracking (step S1160). If a next location exists (step S1170), processing returns to step S1150.

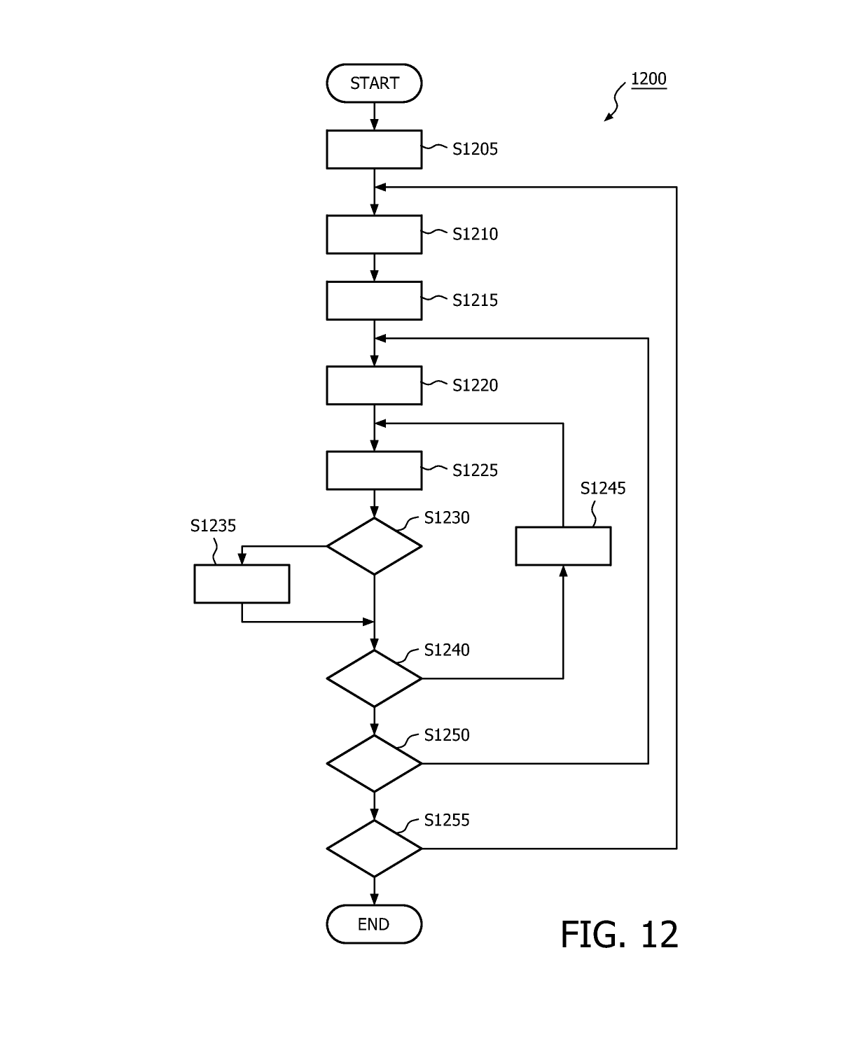

FIG. 12 exemplifies a real-time procedure 1200 for, automatically and without the need for user intervention, time-efficient monitoring of a treatment region 310 exhibiting a certain degree of homogeneity. A baseline 301 is acquired using one or more unfocused pushes 210 each for impinging upon a spatial area within the region 310 wider than that for a focused push. Per each unfocused push, one or more pairs 864 of tracking pulses 215, 220 are issued, the pairs being mutually spaced positionally apart (step S1205). The region 310 is continually scanned, spanning it repeatedly run by run, but skipping recorded locations, the scanning being interrupted, as by expiry of a period of time (step S1210). Logic points to the first unfocused push 210 (step S1215). Logic points to the first location 316 covered by the current unfocused push 210 (step S1220). The current unfocused push 210 issues, followed by the pair 864 of tracking pulses 215, 220 (step S1225). If treatment for the current location 316 is completed (step S1230), the current location is recorded (step S1235). In either case, if there is a next location 316 for tracking the current unfocused push 210 (step S1240) taking into account the skipping of recorded locations 316, beamforming logic steers to scan, i.e., a beam for the unfocused push 210 and the tracking beam 328 of pulses 215, 220, to that next location (step S1245), and processing returns to step S1225. If, on the other hand, tracking of the current unfocused push 210 is completed (step S1240), and there is a next unfocused push (step S1250), processing returns to step S1220. Alternatively, in the event that all unfocused pushes for the treatment region 310 have issued (step S1255), but therapy for the treatment region is not yet completed, processing returns to step S1210.

Energy is transferred to cause a mechanical property of biological tissue to change, as in ablation. An effect of the transferring is examined in more than one spatial dimension to, for example, make an ablation halting decision for a treatment region, i.e., line or layer, or for a location within the region. Halting decisions can be based on lesion-central and/or lesion-peripheral longitudinal displacement of treated tissue evaluated in real time against a characteristic curve. Steering in the azimuthal and/or elevation direction is afforded by, for example, linear, or 2D, multi-channel ultrasound arrays for therapy and imaging. Protocols includable are region-wide scanning and location-by-location completion for both (HIFU) therapy and tracking (acoustic-radiation-forced-based) displacement of treated tissue. Fine, location-to-location monitoring can be used for relatively inhomogeneous tissue; whereas, quicker, sparser and more generalized monitoring can be employed for relatively homogeneous tissue.

In accordance with the present invention, accurate, fast, low-cost, simple and convenient techniques are proposed for real-time, ablation of body tissue in multiple spatial dimensions. A convenient and economical all-ultrasound implementation is afforded, enabling a much more widespread usage of this type of treatment in the United States and emerging markets.

HIFU, being an ultrasound method, affords a low-cost all-ultrasound ablation therapy apparatus with features set forth herein above. Nevertheless, any other form of ablation therapy which likewise causes body tissue to undergo a change in mechanical properties is within the intended scope of the present invention, such as by heating (e.g., radio frequency (RF) ablation, high intensity focused ultrasound (HIFU) ablation, microwave, laser), freezing (e.g., cryogenic ablation) or chemical action.

The present invention is not limited to tumor ablation. The alleviation of cardiac arrhythmia, for example, may be accomplished by necrosing a specific line of heart tissue to thereby block an abnormal electrical path through the heart. Such a method may be accomplished using ablation methods of the present invention.

Moreover, although methodology of the present invention can advantageously be applied in providing medical treatment, the scope of the present invention is not so limited. More broadly, techniques of the present invention are directed to transferring energy to cause a mechanical property of biological tissue in vivo, in vitro or ex vivo to change and to examining, in more than one spatial dimension, an effect of the transferring.

While the invention has been illustrated and described in detail in the drawings and foregoing description, such illustration and description are to be considered illustrative or exemplary and not restrictive; the invention is not limited to the disclosed embodiments.

For example, it is possible to operate the invention in an embodiment wherein the halting decision for a location is based both on real-time observation of the central and one or more peripheral NDD's for the location and histologically-based correlation between lesion size and the respectively offsetted NDDs. Offsetting, can be of pushing and/or tracking, and need not be confined to the periphery, or from the center, of the lesion currently being formed. Also, in another aspect, the electronic steering of the therapy and tracking beams is not limited to discrete locations or to any particular directional protocol.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. Any reference signs in the claims should not be construed as limiting the scope.

A computer program can be stored momentarily, temporarily or for a longer period of time on a suitable computer-readable medium, such as an optical storage medium or a solid-state medium. Such a medium is non-transitory only in the sense of not being a transitory, propagating signal, and thus can be realized as register memory, processor cache or RAM, for example.

A single processor or other unit may fulfill the functions of several items recited in the claims. The mere fact that certain measures are recited in mutually different dependent claims does not indicate that a combination of these measured cannot be used to advantage.

1. A control device (115) for an ablation system that includes an ablation unit (110), the ablation unit including a multi-element diagnostic array (125) placed confocally with a therapy array (130) that issues a therapy beam for transferring energy for changing a mechanical property (304) of biological tissue, said therapy beam having a most recent focus (844), said control device comprising:
a combination multi-channel high power amplifier and matching network module (135);
a triggering and control logic module (140); and
a multi-channel ultrasound data acquisition and analysis module (145),
wherein the triggering and control logic module (140) outputs triggering and control signals to synchronize timing and electronic steering of three types of acoustic beams, including therapy beams, push beams, and tracking beams, that are interspersed,
further wherein the combination multi-channel high power amplifier and matching network module (135) is responsive to triggering and control signals supplied by the triggering and control logic module (140) for applying driving signals to the therapy array (130) to issue an acoustic-radiation-force-based push beam whose focus for assessing an effect of said most recent focus of said therapy beam is, in at least one of an azimuthal and/or elevation direction, offset (830) from said most recent focus of said therapy beam, and
wherein the multi-channel ultrasound data acquisition and analysis module (145) is responsive to triggering and control signals supplied by the triggering and control logic module (140) for electronically steering a tracking beam for displacement monitoring at a particular location that is offset (i) from said most recent focus of said therapy beam in at least one of an azimuthal and/or elevation direction, and (ii) to a target periphery (860) of a lesion being formed by said mechanical-property-changing therapy beam, the tracking being of displacement caused by a push to said biological tissue, in response to said acoustic-radiation-force-based push beam, to assess an effect of the energy transfer by said mechanical-property-changing therapy beam.

2. The control device of claim 1, wherein said offset of said acoustic-radiation-force-based push beam corresponds to the target periphery (860) of a said lesion being created by said mechanical-property-changing therapy beam with said most recent focus.

3. The control device of claim 1, wherein said triggering and control logic module (140) is further configured for outputting triggering and control signals for maintaining said mechanical-property-changing therapy beam (336) at a current location within a treatment region within said biological tissue until said multi-channel ultrasound data acquisition and analysis module (145) issues a halting decision based on a lesion-peripheral longitudinal displacement of treated tissue, corresponding to a peripheral normalized displacement difference (NDD) parameter, against a characteristic curve, determining that treatment at said location is completed.

4. The control device of claim 3, wherein said triggering and control logic module (140) is further configured outputting triggering and control signals for repeatedly interspersing (S930, S940) said mechanical-property-changing therapy beam with said push beam and a tracking beam in real time, and, based on said determining in real time, scanning from said location to a next location within said region in real time.

5. (canceled)

6. The control device of claim 1, wherein said multi-element diagnostic array (125) being two-dimensional and configured for said steering in both said azimuthal (325a) and elevation (325b) directions.

7. The control device of claim 1, wherein said multi-channel ultrasound data acquisition and analysis module (145) is further configured for applying said displacement, in the form of a peripheral normalized displacement difference (NDD) parameter, to a characteristic curve (515) to predict lesion size.

8. The control device of claim 1, wherein the triggering and control logic module (140) is further configured for outputing triggering and control signals to the multi-channel ultrasound data acquisition and analysis module (145) for steering, during an interruption in the energy transfer, the tracking beam from location to location within a treatment region within said biological tissue.

9. The control device of claim 1, wherein said triggering and control logic module (140) is further configured for outputing triggering and control signals to (i) the combination multi-channel high power amplifier and matching network module (135) and (ii) the multi-channel ultrasound data acquisition and analysis module (145) (iii) for creating, prior to introducing thermal effects into a treatment line, or treatment layer, within said biological tissue by means of the energy transfer, a baseline (301) usable in decisions on whether treatment at locations in respectively said line or said layer is completed, said creating being based on results from scanning respectively said line, or said layer, with pushes and tracking pulses.

10. The control device of claim 1, wherein the multi-channel ultrasound data acquisition and analysis module (145) is further configured for determining that a location within a treatment region within said biological tissue is no longer to be treated with a beam by means of which said energy transfer occurs.

11. The control device of claim 10, wherein the triggering and control logic module (140), the combination multi-channel high power amplifier and matching network module (135), and (ii) the multi-channel ultrasound data acquisition and analysis module (145) are further configured for performing in real time said steering, said tracking and said determining.

12. The control device of claim 11, wherein the triggering and control logic module (140), the combination multi-channel high power amplifier and matching network module (135), and (ii) the multi-channel ultrasound data acquisition and analysis module (145) are further configured for performing, automatically and without need for user intervention, said steering, said tracking, said determining, and deciding that treatment of said region is completed.

13. The control device of claim 1, wherein the triggering and control logic module (140) is further configured for outputing triggering and control signals to the combination multi-channel high power amplifier and matching network module (135) for steering a push beam (848) from location to location within a treatment region within said biological tissue during an interruption in said energy transfer.

14. The control device of claim 1, wherein said tracking beam is further offset from said push beam to a target periphery of the lesion (840) currently being formed.

15. The control device of claim 1, wherein the therapy array (130) of said ablation unit comprises a multi-channel ultrasound transducer array configured for steering, in at least one of an azimuthal and elevation direction, a the therapy beam by means of which said energy transfer occurs.

16. (canceled)

17. The control device of claim 10, wherein said triggering and control logic module (140) is further configured for outputing triggering and control signals to (i) the combination multi-channel high power amplifier and matching network module (135) and (ii) the multi-channel ultrasound data acquisition and analysis module (145) (iii) for, while not performing said monitoring, performing said scanning of said region location by location in runs that are repeated, skipping locations for which it has been determined that treatment is completed.

18. The control device of claim 10, wherein said triggering and control logic module (140) is further configured for outputing triggering and control signals to (i) the combination multi-channel high power amplifier and matching network module (135) and (ii) the multi-channel ultrasound data acquisition and analysis module (145) (iii) for, when it is determined that mechanical-property-changing treatment to a current location within said region is no longer to be applied, performing said scanning to a next location if a next location is to be treated, and, without need for any pushing or any tracking, repeating said treatment at said next location which now serves as said current location for purposes of any further repetition.

19. (canceled)

20. A control method for an ablation system that includes an ablation unit, the ablation unit including a multi-element disgnostic array placed confocally with a therapy array that issues a focused therapy beam for transferring energy for causing a mechanical property of biological tissue to change, said therapy beam having a most recent focus, the method comprising:
applying an acoustic-radiation-force-based push beam whose focus (852) for assessing an effect of said most recent focus of said therapy beam is currently, in at least one of an azimuthal and elevation direction, offset from said most recent focus of said therapy beam; and
electronically steering a tracking beam for displacement monitoring at a particular location that is offset (i) from said most recent focus of said therapy beam in at least one of an azimuthal and/or elevation direction, and (ii) to a target periphery of a lesion being formed by said mechanical-property-changing therapy beam, the tracking being of displacement caused by a push to said biological tissue, in response to said acoustic-radiation-force-based push beam, to assess an effect of the energy transfer by said mechanical-property-changing therapy beam.

21. The control method of claim 20, further comprising tracking displacement from said push beam and applying displacement results from said tracking, in the form of a peripheral normalized displacement difference (NDD) parameter, to a characteristic curve to predict lesion size (601).

22. A computer software product for an ablation system that includes an ablation unit, the ablation unit including a multi-element disgnostic array placed confocally with a therapy array that issues a focused therapy beam for transferring energy for causing a mechanical property of biological tissue to change, said therapy beam having a most recent focus, comprising a non-transient computer readable medium embodying a computer program that includes instructions executable by a processor to perform a control method comprising:
applying an acoustic-radiation-force-based push beam whose focus for assessing an effect of the mechanical-property-changing beam of said therapy beam at a current location within a treatment region within said tissue is currently, in at least one of an azimuthal and elevation direction, offset from said most recent focus of said therapy beam; and
applying a tracking beam for displacement monitoring at a particular location whose focus is currently, in at least one of an azimuthal and/or elevation direction, offset (i) from said most recent focus of said therapy beam, and (ii) to a target periphery of a lesion being formed by said mechanical-property-changing therapy beam, the tracking being of displacement caused by a push to said biological tissue, in response to said acoustic-radiation-force-based push beam, to assess an effect of the energy transfer by said mechanical-property-changing therapy beam.
